(19) United States
(12) Patent Application Publication
(10) Pub. No.: US 2025/0114013 A1
(43) Pub. Date: Apr. 10, 2025

(54) LOCATOR DEVICES, SYSTEMS, AND METHODS FOR MEDICAL PROCEDURES

(71) Applicant: BOSTON SCIENTIFIC SCIMED, INC., MAPLE GROVE, MN (US)

(72) Inventors: Jose Roberto Soto Guzman, San Jose (CR); Gian Franco Loo Fuchs, San Jose (CR); Yeison Calvo, San Ramón (CR); Sean P. Fleury, Princeton, MA (US)

(73) Assignee: BOSTON SCIENTIFIC SCIMED, INC., MAPLE GROVE, MN (US)

(21) Appl. No.: 18/910,577

(22) Filed: Oct. 9, 2024

Related U.S. Application Data
(60) Provisional application No. 63/543,312, filed on Oct. 10, 2023.

(51) Int. Cl. A61B 5/06; A61B 5/00

(52) U.S. Cl. CPC A61B 5/065; A61B 5/0084

(57) ABSTRACT

A fiber optics based locator system having a fiber optics portion configured to be advanced directly through a tortuous body passage of a patient without being advanced through the lumen of an independently formed delivery device. A light-emitting element, such as a diffuser, may be provided at the distal end of the system. The fiber optics are selected to transmit light to the light-emitting element capable of being detected through body tissue. For instance, the light-emitting element may be positioned within a first location within a patient, and may be sensed by a sensor positioned within a second location within a patient spaced apart from (e.g., within a different anatomical structure than) the first location. Various procedures may be performed by accessing a target site at or adjacent the first location from a position at or adjacent the second location.

### CROSS-REFERENCE RELATED APPLICATIONS

This application claims the benefit of priority under 35 U.S.C. § 119 to U.S. Provisional Application No. 63/543,312, filed Oct. 10, 2023, the entire disclosure of which is hereby incorporated by reference herein for all purposes.

### FIELD

The present disclosure relates generally to devices, systems, and methods facilitating identification of the location of a target site within a patient's body. More particularly, the present disclosure relates to fiber optics based locator devices and systems which are navigable directly through a body passage without being navigated through a lumen of another device, and methods associated therewith.

### BACKGROUND

Viewing, locating, and manipulating anatomies, devices, and/or anatomies containing one or more devices from within a body may be difficult. During a procedure within the body, such as an endoscopic procedure (not involving cutting open the body), a medical professional may need to externally locate a particular anatomical structure of and/or position within the body. Locating a desired anatomical structure of and/or position within a body may be difficult due to a lack of or low amount of illumination, and/or intervening anatomy, and/or the shape and/or configuration of various portions of the body. For instance, procedures within body lumens such as the intestines present lengthy regions of the anatomy which may be difficult to differentiate from outside the body. Various locator devices may be provided within the body to assist with locating a target site within the body. However, the locator device may need to be navigated through the patient's body to a target site for a procedure. In some instances, the locator device may need to be navigated through tortuous body passages. The locator device may not bend sufficiently, may break, may damage (e.g., puncture, perforate, abrade, etc.) the tissue of the body passage, or may present other challenges during delivery and advancement to a target site within a patient's body. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a fiber optics based locator system includes a fiber optics core; and a jacket over the core. In some aspects, at least the fiber optics core is selected so that the fiber optics based locating system can be directly navigated through tortuous body passages without being damaged and without traumatizing the body passage; and the jacket has an exterior surface configured to facilitate movement of the fiber optics based locating system directly through a body passage.

In some aspects, the fiber optics based locator system further includes a cladding over the fiber optics core, the jacket being positioned over the cladding. In some aspects, the cladding is selected to maintain the integrity of the fiber optics core without interfering with navigability of the fiber optic core through body passages.

In some aspects, the fiber optics based locator system further includes a light-emitting element at a distal end of the fiber optics core. In some aspects, the light-emitting element is a diffuser.

In some aspects, the jacket is provided with a low-friction material to facilitate navigation of the fiber optics based locator system directly through tortuous body passages.

In some aspects, the jacket defines a first lumen through which the fiber optic core extends. In some aspects, the fiber optic core is secured within the first lumen to limit axial translation of the fiber optic core with respect to the lumen. In some aspects, the jacket defines a second lumen for passing the fiber optics based locator system over a guidewire.

In some aspects, the fiber optics based locator system further includes a control handle at a proximal end of the system, the control handle configured to couple a light source with the fiber optics core.

In accordance with various principles of the present disclosure, a system is disclosed for locating a first location within a patient's body from a second location within the patient's body spaced apart from the first location. In some aspects, the system includes a fiber optics based locator system comprising a fiber optics portion with a light-emitting element at a distal end thereof; and a locating system comprising a sensor configured to sense light emitted from the light-emitting element when the fiber optics based locator system is positioned at the first location and the sensor is positioned at the second location. In some aspects, the fiber optics based locator system is configured to be navigated directly through tortuous body passages without extending through a lumen of a separate delivery device.

In some aspects, the fiber optics portion is selected so that the fiber optics based locating system can be directly navigated through tortuous body passages without being damaged and without traumatizing the body passage.

In some aspects, the fiber optics portion includes a fiber optics core and a jacket over the core; and the jacket has an exterior surface configured to facilitate movement of the fiber optics based locating system directly through a body passage. In some aspects, the jacket has an exterior surface configured to facilitate movement of the fiber optics based locating system directly through a body passage.

In some aspects, the fiber optics portion comprises a fiber optics core which transmits light to the light-emitting element; and the fiber optics core is selected to transmit light detectable through a tissue wall. In some aspects, the sensor is selected to detect, through a tissue wall, light emitted by the light-emitting element.

In some aspects, the sensor is selected to detect, through a tissue wall, light emitted by the light-emitting element.

In accordance with various principles of the present disclosure, a method of forming a locating system which is navigable through a tortuous body passage of a patient is disclosed. In some aspects, the method includes forming a fiber optics core from a plurality of fiber optics filaments selected to be sufficiently flexible to navigate tortuous body passages without being damaged and without damaging the body passage; and providing a jacket over the fiber optics core selected from a material which does not affect the flexibility of the fiber optics core and which facilitates navigation of the locating system directly through the body passage without being navigated within a delivery device to reach a first location within the patient's body.

In some aspects, the method further includes providing a lubricious and/or low-friction material with respect to the jacket to facilitate navigation of the locating system through a tortuous body passage with the exterior surface of the jacket exposed to the tortuous body passage.

In some aspects, the method further includes providing a lumen through the jacket configured to pass the jacket and fiber optics core over a guidewire to advance the locating system to the first location.

In some aspects, the method further includes providing a light-emitting element at a distal end of the fiber optics core; and selecting the fiber optics filaments to transmit light to the light-emitting element capable of being sensed from a second location within the patient's body spaced apart from the first location.

In accordance with various principles of the present disclosure, a method of locating a target site adjacent a first location within a patient's body from a second location within the patient's body spaced apart from the first location includes advancing a fiber optics based locating system directly within a body passage within the patient's body so that the exterior surface of the fiber optics based locator system is exposed to the body passage. The method further includes advancing the fiber optics based locator system to a first location within the patient's body. In some aspects, a light-emitting element is provided at a distal end of a fiber optics portion of the fiber optics based locator system, and the method includes advancing the light-emitting element to the first location.

In some aspects, the method includes selecting the fiber optics portion of the fiber optics based locator system to transmit light to the first location which is detectable from the second location. In some aspects, the method includes advancing a locating system to the second location. In some aspects, the locating system includes a sensor selected to be capable of sensing light through body tissue. In some aspects, the sensor is selected to sense light from the first location when the sensor is positioned at the second location.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a passage within a body, and/or a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of a locator system formed in accordance with various aspects of the present disclosure and positioned at a first anatomical location.

FIG. 2 illustrates a view similar to that of FIG. 1, but with the large intestine shown in phantom, and a locating system positioned at a second anatomical location different from the first anatomical location, and configured to locate the locator system at the first anatomical site.

FIG. 3 illustrates a perspective view of an example of an embodiment of a locator system formed in accordance with various principles of the present disclosure.

FIG. 4 illustrates a perspective view of a distal end of an example of an embodiment of a locator system such as in FIG. 3, partially exploded to illustrate the components/layers of the locator element thereof.

FIG. 5 illustrates a perspective view of a section of an example of an embodiment of a locator system such as in FIG. 3, with a portion of the outer layer cut away to illustrate the lumen formed therein.

FIG. 6 illustrates a perspective view of a section of an example of an embodiment of a locator system such as in FIG. 3, with a portion of the outer layer cut away to illustrate the lumens formed therein.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site.

Various medical procedures involve identifying a first location within a body from a second location within the body. For instance, a number of medical procedures require identification of the location of an anatomical structure, such as an organ or a tissue wall, and then delivering medical instruments to such location to perform a procedure on or at the identified location. The identified location may be referenced herein as a “target site” or “target tissue” or “target tissue site”, etc., the present disclosure being applicable to any such location, reference to any one such designation being applicable to the other designations without intent to limit unless otherwise indicated. The second location is typically spaced apart from the first location. In some aspects, the second location is in a different anatomical structure. In some aspects, the first location and the second location are separated by an anatomical wall, such as a wall (e.g., tissue wall) of one or more anatomical structures.

Various devices, systems, and methods provide locating devices (e.g., light beacons) which may be positioned at the first location and sensed (e.g., visualized) at the second location so that a procedure may be performed from the second location with respect to the first location. Non-limiting examples of procedures which may be performed include various medical procedures which involve moving a tissue wall (e.g., a body lumen wall or the wall of an organ) to a desired position, such as relative to another tissue wall (e.g., a body lumen wall or the wall of an organ). For instance, various procedures may be performed by entering the gastrointestinal (GI) tract through a first organ or structure (such as the esophagus, stomach, duodenum, small intestine, large intestine, or peritoneal cavity), and delivering an anchor or stent to adjacent organs or lumen or tissue structures (such as an adjacent portion of the GI tract, the bile duct, the pancreatic duct, the gallbladder, the pancreas, cysts, pseudocysts, abscesses, and the like). Typically, it is necessary to penetrate both an access tissue wall (e.g., a wall of an organ or a first body lumen), through which access is established, and a second tissue wall (e.g., of a wall of an organ or a second body lumen) along or adjacent or at the target for the procedure.

One trend in medicine includes moving from laparoscopic and open surgical procedures to miniaturized, endoscopic procedures. Endoscopists can perform ever more complex procedures noninvasively by inserting a medical device or instrument through a natural orifice (or, in some instances, through a very small incision in the patient). Performance of such procedures within (e.g., exclusively within, without opening or otherwise cutting the body to gain access to the locations within the body) may pose various challenges, including navigating a locating device to the first location within the patient's body. It will be appreciated that terms such as navigate, advance, negotiate, maneuver, etc., and other grammatical forms thereof, may be used interchangeably herein without intent to limit. Moreover, it will be appreciated that navigability of a device or system may be affected by various properties of the device or system contributing to and/or allowing the device or system to advance through tortuous body passages, such as, without limitation, bendability, flexibility, resiliency, etc.

An example of a medical procedure during which a first location within a body is identified from a second location within the body is a procedure for forming an anastomosis between two or more anatomical structures. It will be appreciated that reference herein to an anatomical structure includes, without limitation, organs, passages, lumens, cavities, etc., of a patient. To form an anastomosis between two anatomical structures, a first location within the patient must be identified from a second location within the patient to form a connection and/or to deploy an anastomosis-forming structure (such as a stent) across first and second locations. The anastomosis-forming structure is deployed to extend between the first and second locations and to create an anastomosis therebetween. In some aspects, the first location may be a first anatomical structure, and the second anatomical structure may be the same anatomical structure but at a different spaced apart location from the first location, or a second, different anatomical structure separate and generally spaced apart from the first anatomical structure. Creation of an anastomosis involves bringing the two locations into apposition and maintaining the two structures in apposition. A medical device may form the anastomosis, and, in some instances, the anatomical structures may eventually form an anastomosis from the patient's own tissue.

An example of a procedure involving forming an anastomosis is a gastric bypass procedure in which a gastroenteral anastomosis is formed between the stomach and a portion of the jejunum. In this manner, the stomach's contents (e.g., food and other nutrients) bypass and are not absorbed by the duodenum, and uptake or absorption may be delayed, as such contents travel from the stomach through the small bowel, promoting patient weight loss and possible controlling or resolving type-2 diabetes. Various medical systems may be used to form a gastric bypass. One example of a system/platform includes five devices: a grasper, a locator device, a locating device (for identifying/locating the locator device), a structure for forming an anastomosis, a pyloric closure device. The grasper is configured to grasp a portion of the intestines, and is movable to bring such grasped portion into apposition with the patient's stomach. Typically, the intestines define a first location to which the anastomosis-forming device is to be directed, and the stomach defines a second location from which the anastomosis-forming device is deployed to lead to the first location. The grasper typically is positioned at the second location and is extended to the first location to grasp and pull the anatomical structure defining the first location proximally to the anatomical structure defining the second location. The locator device is typically positioned at the first location and may function as a beacon to assist with identifying/locating the first location from the second location. The locating device is configured to sense the locator device. For instance, the locating device may be a visualization system which can visualize, from the second location, the locator device at the first location. The locating device may be a component of a delivery device (e.g., an endoscope) with which the grasper is delivered, or may be delivered independently of the grasper. The device for forming an anastomosis may be a tubular element, such as a stent, configured to extend from the patient's stomach to a portion of the patient's intestines downstream from the pyloric bulb (e.g., downstream from the proximal portion of the duodenum). Typically, the anastomosis-forming device extends from the patient's stomach to a target site along the patient's jejunum, such as approximately 150 cm from the pylorus. The pyloric closure device is configured to be deployed and maintained in position across the patient's pylorus. Moreover, the pyloric closure device is configured to occlude the pylorus and to be maintained in position across the pylorus (e.g., to resist migration forces such as from typical biological gastrointestinal functions) so that materials from the stomach are redirected from the pylorus to the anastomosis.

During a typical gastric bypass procedure, the physician needs to locate a segment of the jejunum approximately 150 cm distal to the pylorus. A locator device, such as a light beacon, is introduced into the small intestines, and tracked until it reaches the target site for the bypass. A scope, such as an endoscope, may be used to introduce the locator device. The ultimate position of the locator device (referenced herein as a “first location”) is at or adjacent to the target location for the bypass. The bypass or anastomosis formed by the procedure (specifically, by the anastomosis-forming device which is positioned to bypass a portion of the duodenum) is a bypass of the duodenum up to the target site, formed by the anastomosis between the stomach and the target site. The bypass/anastomosis may be alternately referenced herein as a gastrojejunostomy or GJ.

Current designs of locator elements may include a guidewire with a light source attached at or adjacent a distal end thereof. The physician performing the procedure typically introduces a scope through the pylorus and then passes the guidewire light source into the small intestines until it reaches the target position. Currently, one of the biggest challenges with available locator devices is the ability to successfully navigate the device to the first location and target site. Various challenges to the ability of a device to be navigated include, without limitation, pushability and trackability of the device. For instance, the tortuosity of the small intestines, and variation between anatomies of patients, create tortuous body passages with relative small radii of curvature. Moreover, the tissue of such body passages may be sensitive and may lead to significant health risks if damaged or perforated or punctured by a device navigated therein.

In accordance with various principles of the present disclosure, to address the above-described challenges of delivering a locating device transluminally to a target site within a body, a fiber optics based device is used as a locator device. Typically, in the field of endoscopy, particularly in transluminal procedures, fiber optics are used within an endoscope, but do not generally have the necessary pushability as well as bendability/flexibility to be able to navigate through tortuous body passages on their own (e.g., independently of a separate delivery device, such as a separate tubular delivery element such as an endoscope). In accordance with various principles of the present disclosure, a fiber optics based locating devices and systems sized, shaped, configured, and/or dimensioned to be pushable as well as navigable on its own (without a separate delivery device through which the fiber optics device is navigated) through tortuous body passages (e.g., through the small intestines) without other performance issues which may occur with currently available locator devices and systems. Common performance issues with currently available locator device and systems include, without limitation, turning back or looping on itself, kinking, cracking (including microscopic fissures which may affect performance of the locating device), breaking, and/or damaging (e.g., scratching, abrading, perforating, puncturing, etc.) the wall of the body passage. Additionally or alternatively, a fiber optics based locator device and system is formed in accordance with various principles of the present disclosure to be readily advanceable (e.g., movable, slidable, etc.) within a patient's body. In some aspects, a fiber optics based locator device and system formed in accordance with various principles of the present disclosure has a lubricious and/or low-friction exterior/outer surface, to facilitate navigation thereof directly through a tortuous body passage (and not through a lumen of another device inserted into the body passage). As such, a fiber optics based locator device and system formed in accordance with various principles of the present disclosure addresses various shortcomings of prior locator devices and systems, and also addresses various challenges presented by navigating a fiber optics device through a tortuous body passage independently of a separate delivery device.

In accordance with various principles of the present disclosure, pushability and looping issues commonly associated with locator devices and systems inserted into tortuous body passages are significantly reduced by using a fiber optics based system formed in accordance with various principles of the present disclosure. The fiber optics device of the present disclosure optionally includes a diffuser/projector at/in the distal tip of the device that is configured to aim the light of the fiber optics to the target site at or adjacent a first location to be identified at a second location. In some aspects, a fixed or detachable handle contains or carries the light source, and the fiber optics conducts the light to a diffuser at the distal tip of the device or system. The diffuser outputs light from the fiber optics, such as to project the light from the fiber optics to the first location, in order to allow the physician to locate and identify the first location from a second location, and to locate and grasp accurately tissue at the first location (e.g., a target location) from a position at the second location.

In some aspects, a jacket is provided over the fiber optics portion of the system. In some aspects, the jacket is formed of a material which will provide, or is otherwise treated and/or coated to provide, a low friction and/or lubricious exterior surface for the fiber optics based locator device and system. The exterior surface of the fiber optics based locator device and system of the present disclosure thus may readily navigate through tortuous body passages without causing trauma to tissue, organs, etc. Additionally or alternatively, the jacket is configured to contain/retain the light inside the fiber optics. In some aspects, the diffuser is the only component of the fiber optics that allows passage of light so that the light passing through the fiber optics is visible from a second location spaced apart from the first location at which the diffuser is positioned. In some aspects, a cladding is provided over the fiber optics core of the fiber optics portion of the fiber optics based locator system. The fiber optic core may be a clear core. The cladding may be formed of a material which contains/retains the light within the fiber optic core (e.g., may be formed of borosilicate, clear polymers, etc.) positioned over (e.g., enveloping, covering, containing, etc.) the fiber optics core. In some aspects, the cladding is selected to provide the environmental requirements for substantially total internal reflection of light within the fiber optic core. In some aspects, the cladding is covered by a single or dual lumen extrusion (HDPE, polyimide, etc.). It will be appreciated that such extrusion may form the jacket component of the fiber optics portion of the fiber optics based locator system. In embodiments of extrusions with two lumens, the first lumen is configured for placement of the fiber optics core, as a jacket, and the second lumen may be used to navigate the fiber optics based device over a guidewire (e.g., by passing the guidewire through the second lumen, and then guiding the fiber optics based device over the guidewire) until the device reaches the target location. If a single lumen extrusion is provided, then a coil and/or braided jacket or other reinforcing material or member, may be added to the single lumen to increase the stiffness profile of the device as desired and/or indicated such as by the nature of the procedure. Radiopaque markers can be added over the extrusion to facilitate visualization of the device with fluoroscopy and/or to confirm that the device is not looping or otherwise not advancing as desired.

Various embodiments of a fiber optics based devices and systems, and associated methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc., in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, certain features in one embodiment may be used across different embodiments and are not necessarily individually labeled when appearing in different embodiments.

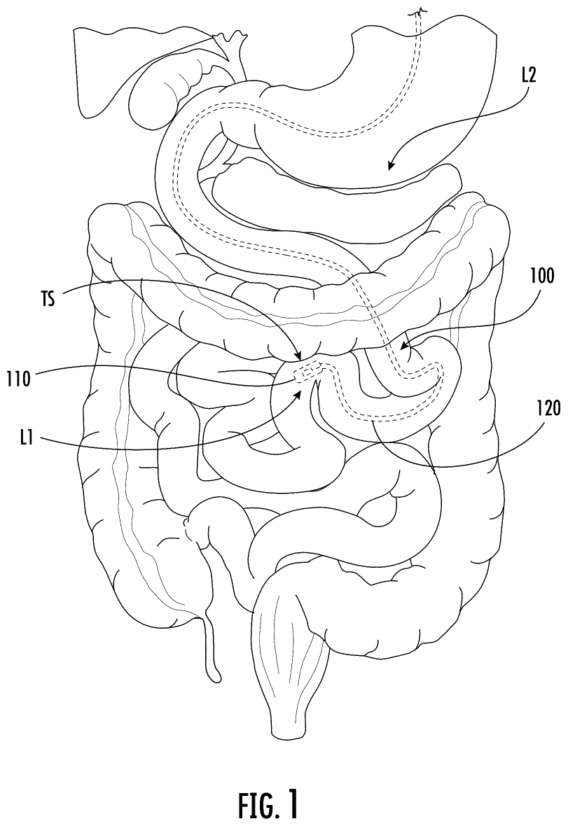

Turning now to the drawings, an example of an embodiment of a fiber-optics based locator system 100 formed in accordance with various principles of the present disclosure is illustrated in FIG. 1 in a schematic representation of a gastrointestinal system. It will be appreciated that principles of the present disclosure may be applied to other anatomical sites and structures, reference being made to gastrointestinal locations and structures for the sake of convenience and without intent to limit. In the illustrated example of an environment, a fiber optics based locator system 100 may be inserted, such as through a natural orifice transluminal endoscopic surgery (NOTES) procedure (e.g., through the nose or mouth, and into the esophagus), into and through the stomach S, through the pylorus P, and into the duodenum D. In some embodiments, the fiber optics based locator system 100 is inserted through a flexible elongate delivery member (e.g., a shaft, catheter, endoscope, etc.), such as known to those of ordinary skill in the art for transluminal delivery of devices through the body (in contrast with via open-surgery techniques). Once the fiber optics based locator system 100 is within the patient's body, the portion of the fiber optics based locator system 100 which may be identified by a locating system may be advanced to a first location L1. For instance, the illustrated example of an embodiment of a fiber optics based locator system 100 emits light at a light-emitting element 110, such as a diffuser/projector, described in further detail below. In some embodiments, the fiber optics based locator system 100 is configured to direct light transmitted through a fiber optics portion 120 of the system 100 to the light-emitting element 110 positioned at a target site TS. The light-emitting element 110 is advanced to the first location L1, and may be identified (sensed, detected, etc.) from a second location L2 spaced apart from (e.g., in a different anatomical structure from) the first location L1, such as by a locating device positioned at the second location L2, as described in further detail below. In some embodiments, one or more visualization markers, such as radiopaque markers, bands, or radiopaque filler materials, may be provided over the fiber optics based locator system 100 to facilitate visualization of the fiber optics based locator system 100, such as during navigation to the target site TS and or in locating the fiber optics based locator system 100 from the second location L2.

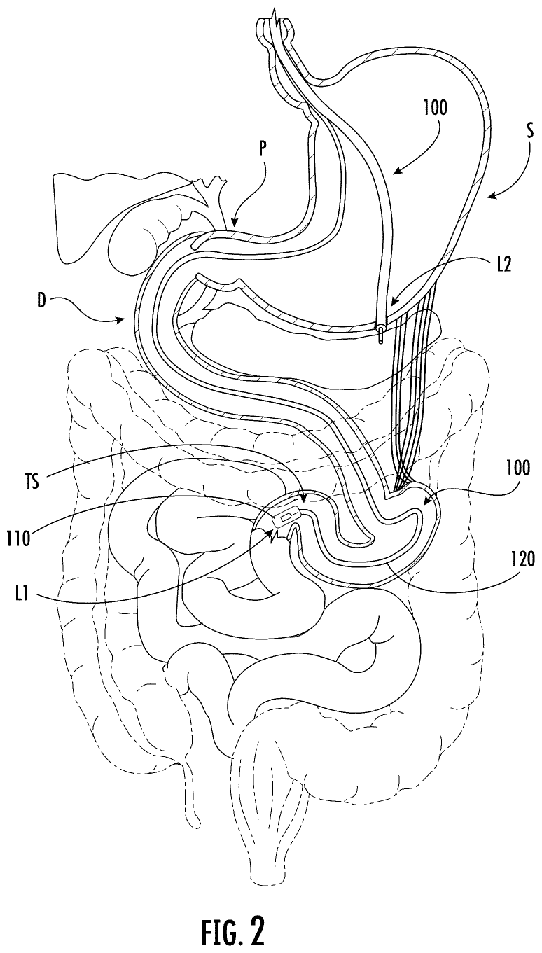

The first location L1 typically is adjacent, in the vicinity of, at, etc., a target site TS within the patient's body. As referenced herein, the target site TS is an anatomical site at or with respect to which a procedure is to be performed, such as from the second location L2. The first location L1 may be coextensive with or spaced apart from the target site TS, but in any case is sufficiently adjacent the target site TS such that identification of the first location L1 (at which the light-emitting element 110 of the fiber optics based locator system 100 is positioned) with a locating system positioned at the second location L2 allows a procedure to be performed from the second location L2 with respect to the target site TS. For instance, further devices or systems may be delivered to the second location L2, such as with the assistance of a flexible elongate delivery member 200, to identify/locate the fiber optics based locator system 100 and/or to perform a procedure with respect to the first location L1, as illustrated schematically in FIG. 2.

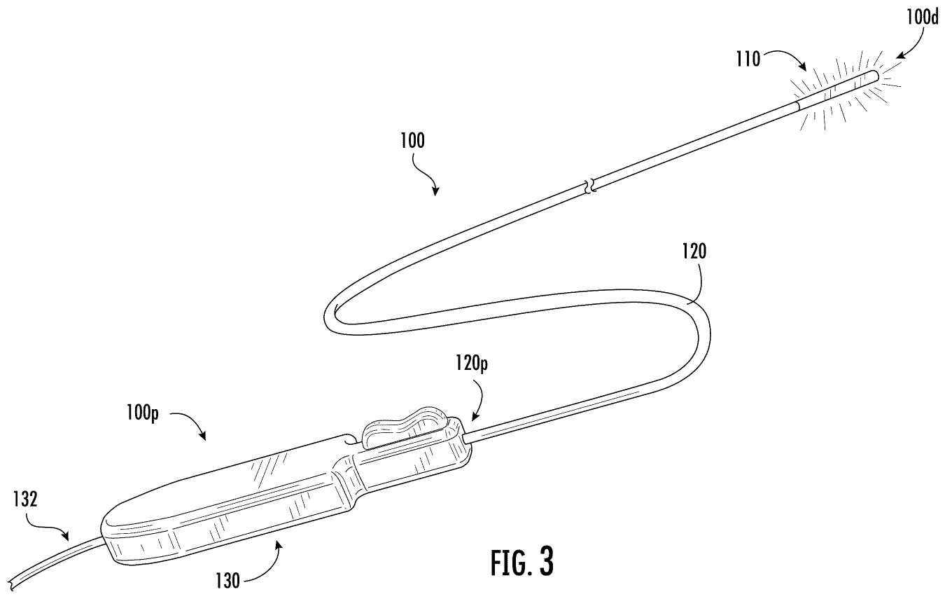

In accordance with various principles of the present disclosure, and as described in further detail below, the fiber optics based locator system 100 is formed (e.g., sized, shaped, configured, and/or dimensioned) to be advanceable through tortuous pathways within the patient's body without kinking, looping or turning back on itself, breaking, damaging tissue, or otherwise not performing as intended. In the example of an embodiment illustrated in FIG. 3, the locator system 100 includes a light-emitting element 110 at a distal end 100d thereof, and a control handle 130 at a proximal end 100p thereof. In some aspects, positioning of the light-emitting element 110 at a distalmost end of the fiber optics based locator system 100 allows the fiber optics based locator system 100 to be positioned at a first location L1 adjacent to yet spaced apart from the target site TS so that the fiber optics based locator system 100 does not interfere with access to the target site TS by a medical instrument extending thereto from the second location L2. The control handle 130 may be operated by a medical professional to advance the light-emitting element 110 of the fiber optics based locator system 100 to the first location L1. Additionally or alternatively, the control handle 130 may be configured to contain, support, carry, or otherwise couple a light and/or energy source to the fiber optics portion 120 of the fiber optics based locator system 100. Light or energy coupled to the fiber optics portion 120 via the control handle 130 may be transmitted through the longitudinal extent of the fiber optics portion 120 to provide illumination at or adjacent the distal end 100d of the fiber optics based locator system 100, such as to the light-emitting element 110. Additionally or alternatively, the control handle 130 may include a connector 132 via which power may be supplied to the fiber optics based locator system 100. Additionally or alternatively, the control handle 130 may be removably coupled to the proximal end 120p of the fiber optics portion 120, such as to allow removal of the control handle 130, such as for backloading of the fiber optics based locator system 100 into the distal end of another medical device or system.

In some embodiments, a flexible elongate delivery member (e.g., catheter, shaft, medical scope such as an endoscope, etc.) may facilitate delivery of the fiber optics based locator system 100 into a patient. In some aspects, after facilitate delivery of the fiber optics based locator system 100, the flexible elongate delivery member may be moved to another position within the patient's body to facilitate delivery of a locating system at a second location L2 within the patient's body spaced apart from the first location L1 at which the fiber optics based locator system 100 is positioned. For instance, a flexible elongate delivery member 200, such as illustrated in FIG. 2, may be used to facilitate delivery of the fiber optics based locator system 100 into a patient's body, and then may be repositioned to a second location L2 within the patient's body. The second location L2 is spaced apart from the first location L1 to which the fiber optics based locator system 100 is advanced after delivery, and may be in a separate anatomical structure. For instance, in the example of an embodiment of an anatomical environment illustrated in FIG. 2, the fiber optics based locator system 100 is delivered to a first location L1 within the patient's small intestines (e.g., within the jejunum, such as approximately 150 cm from the pylorus), and the flexible elongate delivery member 200 is positioned at a second location L2 within the patient's stomach. However, a fiber optics based locator system 100 and/or flexible elongate delivery member 200 formed in accordance with various principles of the present disclosure may be delivered to other anatomical structures without limitation. The illustrated example of an embodiment of a flexible elongate delivery member 200 may have visualization and/or imaging capabilities, or may deliver a separate instrument with such capabilities, to the second location L2 to facilitate identifying/locating a target site TS to which the fiber optics based locator system 100 has been advanced. Light emitted by the light-emitting element 110 facilitates identification of the first location L1 from a second location L2 within the patient's body. More particularly, light from the light-emitting element 110 is detectable by a sensing element positioned at the second location L2 (e.g., a part of the flexible elongate delivery member 200 or a part of an instrument delivered by the flexible elongate delivery member 200). The sensing element may be a light sensing element, a camera, or other element capable of detecting light from the fiber optics based locator system 100 and indicating such detection to a medical professional so that the medical professional may identify the first location L1 and the target site TS. In some non-limiting embodiments, the flexible elongate delivery member 200 is an endoscope with a camera capable of detecting light emitted by a light-emitting element 110 of the fiber optics based locator system 100.

As discussed above, a fiber optics based locator system 100 is formed in accordance with various principles of the present disclosure to be sufficiently flexible to navigate tortuous pathways within a patient's body without kinking, looping or turning back on itself, breaking, damaging tissue, or otherwise not performing as intended. In accordance with various principles of the present disclosure, various challenges typically presented by the use of fiber optics in meeting such criteria for a fiber optics based locator system 100 have been overcome by various features, characteristics, properties, etc., of the fiber optics portion 120 of the fiber optics based locator system 100 illustrated in FIG. 3. For instance, one or more of the components of the fiber optics based locator system 100 may contribute to optimizing the performance/capability of the fiber optics portion 120 of the fiber optics based locator system 100 of the present disclosure.

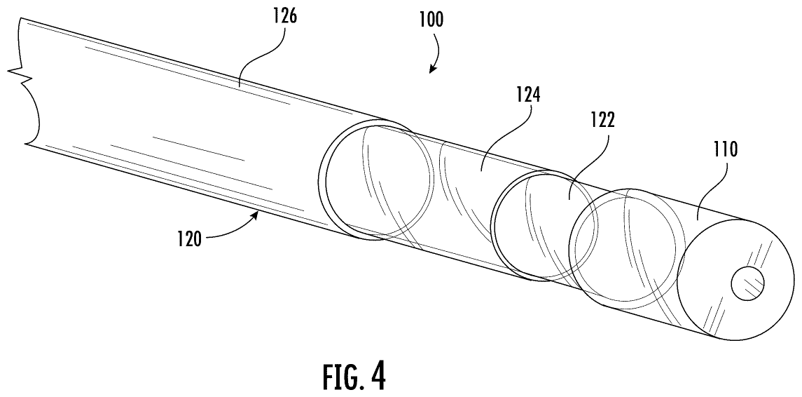

An example of an embodiment of various components of a fiber optics portion 120 of a fiber optics based locator system 100 formed in accordance with various principles of the present disclosure is illustrated in FIG. 4 to facilitate description of a fiber optics based locator system 100 formed in accordance with various principles of the present disclosure, but not to otherwise limit the present disclosure. The illustrated example of an embodiment of a fiber optics portion 120 of a fiber optics based locator system 100 is shown as having more than one component. More particularly, the illustrated example of an embodiment of a fiber optics portion 120 includes a fiber optic core 122, a cladding 124 over the fiber optic core 122, and a jacket 126. In some aspects, the light-emitting element 110 is considered a component of the fiber optics portion 120 as well. In some embodiments, one or more visualization markers 102, such as radiopaque markers, bands, or radiopaque filler materials, are provided over the jacket 126 to facilitate visualization of the fiber optics based locator system 100, such as during navigation to the target site TS and or in locating the fiber optics based locator system 100 from the second location L2.

In accordance with various principles of the present disclosure, one or more of the various components 122, 124, 126 of the fiber optics portion 120 of a fiber optics based locator system 100 (and optionally also the light-emitting element 110) may contribute to the ability of the fiber optics based locator system 100 of the present disclosure to perform as desired. For instance, the fiber optic core 122 may be formed from a plurality of individual light-transmitting fibers/filaments, with one or more of such light-transmitting fibers having a diameter selected to be sufficiently small to achieve a small enough bending radius to navigate through tortuous body passages (e.g., a bending radius of less than about 3″ or 7.62 cm). In some aspects, the diameter of the fiber optic core 122 is approximately 0.025″ (0.635 mm). In some aspects, the diameters of the various individual light-transmitting fibers which compose/comprise/form the fiber optic core 122 are selected to achieve the desired bending radius while having sufficient strength to not crack and/or to not otherwise deform or be damaged, while also not kinking, or looping or turning back on itself. Additionally or alternatively, the light-transmitting fibers should be capable of transmitting light such that the light at the light-emitting element 110 is detectable at a second location L2 spaced apart from the location of the light-emitting element 110, and potentially through a tissue wall. In some aspects, the light-transmitting fibers are selected to transmit short wavelength at least about 850 nm and/or at most about 1300 nm. In some aspects, the light-transmitting fibers are multi-mode glass fibers. In some aspects, a sufficient number of light-transmitting fibers are used to transmit a sufficient amount of light at a sufficient intensity and/or frequency to be detected from a second location L2 spaced apart from the first location L1 at which the light-emitting element 110 is positioned, while not impeding the navigability of the fiber optic core 122 (e.g., formed by the aggregate of light-transmitting fibers). It will be appreciated that the characteristics of the light-transmitting fibers and/or the fiber optic core 122 are selected so that a sufficient amount, brightness, intensity, luminosity, etc., of light is delivered to the light-emitting element 110 of the fiber optics based locator system 100 at the first location L1 to be detected at a second location L2 which may be in a different anatomical structure. In other words, the light must be able to be sensed through one or more anatomical walls between the first location L1 and the second location L2.

In some aspects, the cladding 124 of the fiber optics portion 120 is selected so as to maintain the structure of the fiber optic core 122 without affecting the navigability/flexibility/bendability of the fiber optic core 122. As such, the cladding 124 may have sufficient strength to maintain the structure (e.g., diameter, integrity, etc.) of the fiber optic core 122 without affecting the flexibility of the fiber optic core 122 and the fiber optics portion 120 of the fiber optics based locator system 100 in general. In some aspects, the cladding 124 is formed of a material such as polytetrafluoroethylene (PTFE), fluorinated ethylene propylene (FEP), etc.

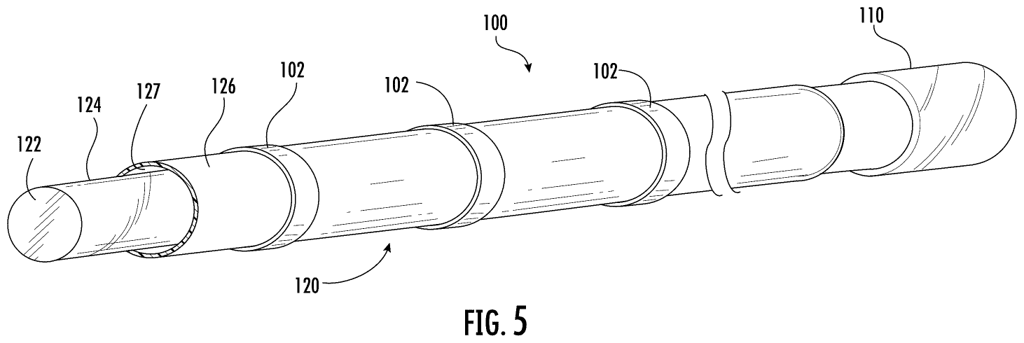

In the example of an embodiment illustrated in FIG. 4 and FIG. 5, the jacket 126 may be considered as a jacket or covering over the cladding 124 and fiber optic core 122, and bonded thereto. The combined components of the fiber optic core 122, cladding 124, and jacket 126 form a fiber optics portion 120 with the necessary characteristics to be able to be navigated through tortuous body passages to be advanced to the target site TS. In accordance with various principles of the present disclosure, the jacket 126 is the outermost component of the fiber optics portion 120 of the fiber optics based locator system 100. In other words, the fiber optics based locator system 100 of the present disclosure is designed to be inserted, advanced, and navigated through tortuous passages within a patient's body directly, and not through another separately formed tubular element. As such, the jacket 126, or another outermost layer or component of the fiber optics portion 120 if provided, may directly contact the inner walls of the tortuous body passages. In accordance with various principles of the present disclosure, the jacket 126 of the fiber optics portion 120 of the fiber optics based locator system 100 is configured to facilitate navigation of the fiber optics based locator system 100 to the target site TS. For instance, the jacket 126 may be formed or coated or otherwise provided with a low-friction and/or lubricious biocompatible material facilitating navigation of the fiber optics portion 120 with respect to body tissue as the fiber optics portion 120 is navigated through a body passage. More particularly, the jacket 126 may form the outermost or exterior-most surface of the fiber optics based locator system 100 (or at least the fiber optics portion 120 thereof) and may thus be configured to facilitate navigability of the fiber optics based locator system 100 by presenting a low-friction and/or lubricious surface of the fiber optics based locator system 100 to the body passage to facilitate movement with respect to. In some aspects, the example of an embodiment of a fiber optics portion 120 of a fiber optics based locator system 100 illustrated in FIG. 5 may be considered to have a single-lumen jacket 126 with a lumen 127 defined therethrough. The fiber optic core 122 (and cladding 124) may be secured within the lumen 127, such as with an adhesive or welding or bonding, such as to maintain the integrity of the fiber optics portion 120 for proper/efficient transmission of light therethrough, and/or to limit axial translation with respect to the lumen 127, and/or to facilitate advancement of the fiber optics portion 120 as a unit. It will be appreciated that a portion of the jacket 126 in the example of an embodiment illustrated in FIG. 5 is shown removed to facilitate illustration of the lumen 127 therein.

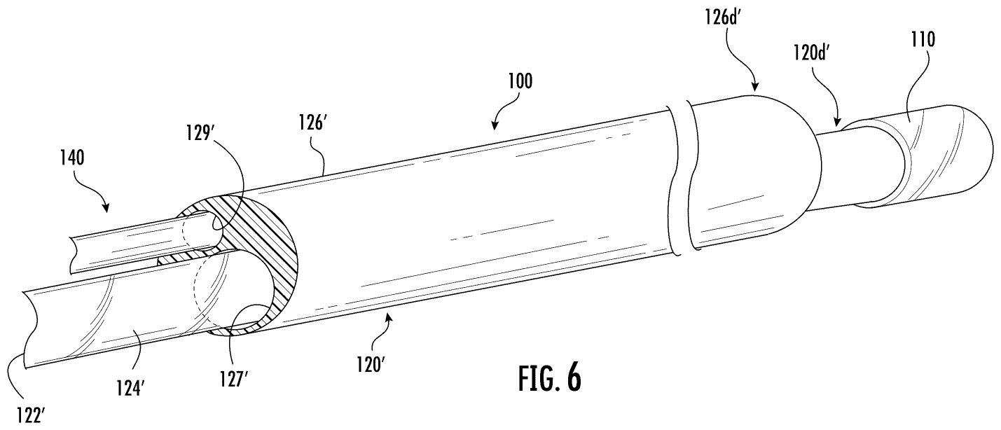

Although the example of an embodiment of a fiber optics based locator system 100 illustrated in FIG. 4 and FIG. 5 is navigated to a target site TS within a patient's body independently of another separately formed element, in some embodiments a fiber optics based locator system formed in accordance with various principles of the present disclosure may be navigated with the assistance of a separately formed element such as a guidewire. For instance, in some aspects, it may be desirable to use a guidewire to facilitate advancement of a fiber optics based locator system 100 to a target site. In such instance, the outermost surface of the fiber optics based locator system is still, at least for the most part, the outermost surface of the device/system which is being navigated. However, the interior of the fiber optics based locator system 100 is configured to accommodate/be guided over a guidewire. In accordance with various principles of the present disclosure, to facilitate navigation of a fiber optics based locator system formed in accordance with various principles of the present disclosure with the assistance of a guidewire, the fiber optics based locator system may be provided with a guidewire lumen through which the guidewire is passed and with which the fiber optics based locator system is guided over the guidewire. In another example of an embodiment of a fiber optics portion 120′ of a fiber optics based locator system 100 formed in accordance with various principles of the present disclosure, such as illustrated in FIG. 6, the jacket 126′ portion is a dual-lumen jacket 126′. It will be appreciated that a portion of the jacket 126′ in the example of an embodiment illustrated in FIG. 6 is shown removed to facilitate illustration of the two lumen 127′, 129′ therein. It will further be appreciated that other than the lumens 127, 127′, 129′, the components of the examples of embodiments of fiber optics portion 120, 120′ illustrated in FIG. 5 and FIG. 6 can be arranged and operate in substantially the same or similar manners. Accordingly, for the sake of brevity and convenience, and without intent to limit, common elements with common functions are indicated with the same reference characters or reference characters followed by a ‘, reference being made to the above descriptions of similar elements and operations. Moreover, it will be appreciated that a fiber optics based locator system 100 with a fiber optics portion 120’ such as illustrated in FIG. 6 may otherwise have components, features, etc., as illustrated in FIGS. 1-5, other than the jacket of the fiber optics portion illustrated in such figures. As such, reference is made to the description of other components, features, etc., of a fiber optics based locator system 100 as described with respect to the fiber optics portion 120 illustrated in FIG. 4 and FIG. 5, and further description of other components, features, etc., of a fiber optics based locator system 100 having a fiber optics portion 120′ as illustrated in FIG. 6 is omitted for the sake of brevity, without intent to limit.

In the example of an embodiment of a fiber optics portion 120′ illustrated in FIG. 6, the fiber optic core 122′ (and cladding 124′) extends through the first lumen 127′, and optionally arc secured therein, such as with an adhesive or welding or bonding. Optionally, a distal end 120d′ of the fiber optics portion 120′ includes a distal portion of the fiber optic core 122′ and cladding 124′ extending beyond the jacket 126′ so that the dimensions of the light-emitting element 110 need not be increased to correspond with the increased dimensions of the jacket 126′ (to accommodate the second lumen 129′ for the guidewire 140). Alternatively, the light-emitting element 110 may be mounted on the distal end 126d′ of the jacket 126′.

In contrast with the bond between the first lumen 127′ and the fiber optic core 122′, the second lumen 129′ of the jacket 126′ of the example of an embodiment of a fiber optics portion 120′ illustrated in FIG. 6 is configured to allow a guidewire 140 to be freely movable therethrough/with respect thereto. In some aspects, the inner diameter of the second lumen 129′ is selected based on typical outer diameters of guidewires so as to allow free passage of common guidewires therethrough. In some aspects, the interior of the second lumen 129′ may be coated or otherwise provided with a low-friction and/or lubricious material which facilitates relative movement between the guidewire 130 and the second lumen 129′ of the jacket 126′ so that the fiber optics based locator system 100 (and, particularly, the second lumen 129′) may be advanced over the guidewire 140 to a target site within the patient's body. More particularly, in some aspects, the guidewire 140 is advanced to a target site within the patient's body, and then the fiber optics based locator system 100 is guided over the guidewire 140 (e.g., by backloading a proximal end of the guidewire 140 into a distal opening of the second lumen 129′) and the light-emitting element 110 is advanced to the target site. In some aspects, a light source is activated (e.g., via the control handle 130) to supply light to the light-emitting element 110 after the light-emitting element 110 has been delivered to the target site. In some aspects, the control handle 130 of the fiber optics based locator system 100 may be removable (such as described above) to allow for removal of the guidewire 140 once the light-emitting element 110 has been positioned at the selected target site TS.

In view of the above, it will be appreciated that a fiber optics based locator system 100 is provided in accordance with various principles of the present disclosure with a small profile (e.g., small outer diameter relative to existing locator systems) and with sufficient flexibility and durability to be navigated through tortuous body passages. As discussed above, one or more components of the fiber optics based locator system 100 may be sized, shaped, configured, and/or dimensioned or otherwise formed to facilitate, contribute, optimize, etc., the desired characteristics of the fiber optics based locator system 100. It will be appreciated that the illustrations of the one or more components of examples of embodiments of fiber optics portion 120, 120′ of a fiber optics based locator system 100 depicted in FIG. 5 and FIG. 6 are not necessarily shown to scale and/or may be otherwise modified and/or exaggerated to illustrate the arrangements of such components.

Although embodiments of the present disclosure may be described with specific reference to medical devices and systems and procedures for treating the gastrointestinal system, it should be appreciated that such medical devices, systems, and methods may be used to treat tissues of the abdominal cavity, digestive system, urinary tract, reproductive tract, respiratory system, cardiovascular system, circulatory system, and the like. References to gastrojejunostomies are solely for the sake of example, without intent to limit. Moreover, although embodiments of the present disclosure may be described with specific reference to medical devices and systems (e.g., endoscopic devices, accessory tools, and/or guidewires) used in the GI system, it should be appreciated that locator devices, systems, and methods as described herein may be used with a variety of medical procedures performed in ductal, luminal, vascular, or other body lumen anatomies, including, for example, interventional radiology procedures, balloon angioplasty/angiography procedures, thrombolysis procedures, urological or gynecological procedures, and the like. The disclosed medical devices and systems may also be inserted via different access points and approaches, e.g., percutaneously, endoscopically, laparoscopically, or combinations thereof.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

Various further benefits of the various aspects, features, components, and structures of locating devices, systems, or methods such as described above, in addition to those discussed above, may be appreciated by those of ordinary skill in the art. The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A fiber optics based locator system comprising:
a fiber optics core; and
a jacket over said core;
wherein:
at least said fiber optics core is selected so that said fiber optics based locating system can be directly navigated through tortuous body passages without being damaged and without traumatizing the body passage; and
said jacket has an exterior surface configured to facilitate movement of said fiber optics based locating system directly through a body passage.

2. The fiber optics based locator system of claim 1, further comprising a cladding over said fiber optics core, said jacket being positioned over said cladding, wherein said cladding is selected to maintain the integrity of said fiber optics core without interfering with navigability of said fiber optic core through body passages.

3. The fiber optics based locator system of claim 1, further comprising a light-emitting element at a distal end of said fiber optics core.

4. The fiber optics based locator system of claim 3, wherein said light-emitting element is a diffuser.

5. The fiber optics based locator system of claim 1, wherein said jacket is provided with a low-friction material to facilitate navigation of said fiber optics based locator system directly through tortuous body passages.

6. The fiber optics based locator system of claim 1, wherein said jacket defines a first lumen through which said fiber optic core extends.

7. The fiber optics based locator system of claim 6, wherein said fiber optic core is secured within said first lumen to limit axial translation of said fiber optic core with respect to said lumen.

8. The fiber optics based locator system of claim 6, wherein said jacket defines a second lumen for passing said fiber optics based locator system over a guidewire.

9. The fiber optics based locator system of claim 1, further comprising a control handle at a proximal end of said system, said control handle configured to couple a light source with said fiber optics core.

10. A system for locating a first location within a patient's body from a second location within the patient's body spaced apart from the first location, said system comprising:
a fiber optics based locator system comprising a fiber optics portion with a light-emitting element at a distal end thereof; and
a locating system comprising a sensor configured to sense light emitted from said light-emitting element when said fiber optics based locator system is positioned at the first location and said sensor is positioned at the second location;
wherein said fiber optics based locator system is configured to be navigated directly through tortuous body passages without extending through a lumen of a separate delivery device.

11. The system of claim 10, wherein said fiber optics portion is selected so that said fiber optics based locating system can be directly navigated through tortuous body passages without being damaged and without traumatizing the body passage.

12. The system of claim 10, wherein:
said fiber optics portion comprises a fiber optics core and a jacket over said core; and
said jacket has an exterior surface configured to facilitate movement of said fiber optics based locating system directly through a body passage.

13. The system of claim 12, wherein said jacket has an exterior surface configured to facilitate movement of said fiber optics based locating system directly through a body passage.

14. The system of claim 10, wherein:
said fiber optics portion comprises a fiber optics core which transmits light to said light-emitting element; and
said fiber optics core is selected to transmit light detectable through a tissue wall.

15. The system of claim 14, wherein said sensor is selected to detect, through a tissue wall, light emitted by said light-emitting element.

16. The system of claim 10, wherein said sensor is selected to detect, through a tissue wall, light emitted by said light-emitting element.

17. A method of forming a locating system which is navigable through a tortuous body passage of a patient, said method comprising:
forming a fiber optics core from a plurality of fiber optics filaments selected to be sufficiently flexible to navigate tortuous body passages without being damaged and without damaging the body passage; and
providing a jacket over the fiber optics core selected from a material which does not affect the flexibility of the fiber optics core and which facilitates navigation of the locating system directly through the body passage without being navigated within a delivery device to reach a first location within the patient's body.

18. The method of claim 17, further comprising providing a lubricious and/or low-friction material with respect to the jacket to facilitate navigation of said locating system through a tortuous body passage with the exterior surface of the jacket exposed to the tortuous body passage.

19. The method of claim 17, further comprising providing a lumen through said jacket configured to pass the jacket and fiber optics core over a guidewire to advance the locating system to the first location.

20. The method of claim 17, further comprising:
providing a light-emitting element at a distal end of the fiber optics core; and
selecting the fiber optics filaments to transmit light to the light-emitting element capable of being sensed from a second location within the patient's body spaced apart from the first location.
